SEQUENCE LISTING (11 sequences)

SEQ ID NO: 1 - protein, 458 aa, Stevia rebaudiana
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 2 - protein, 458 aa, Artificial Sequence
  Synthetic
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQIAKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 3 - protein, 458 aa, Artificial Sequence
  Synthetic
MNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSLENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYS

SEQ ID NO: 4 - protein, 475 aa, Artificial Sequence
  Synthetic
MNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSLYKDDSGYSSSYAAAAGMENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYS

SEQ ID NO: 5 - protein, 462 aa, Oryza sativa
MDSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDVFHHWAAAAALEHKVPCAMMLLGSAHMIASIADRRLERAETESPAAAGQGRPAAAPTFEVARMKLIRTKGSSGMSLAERFSLTLSRSSLVVGRSCVEFEPETVPLLSTLRGKPITFLGLMPPLHEGRREDGEDATVRWLDAQPAKSVVYVALGSEVPLGVEKVHELALGLELAGTRFLWALRKPTGVSDADLLPAGFEERTRGRGVVATRWVPQMSILAHAAVGAFLTHCGWNSTIEGLMFGHPLIMLPIFGDQGPNARLIEAKNAGLQVARNDGDGSFDREGVAAAIRAVAVEEESSKVFQAKAKKLQEIVADMACHERYIDGFIQQLRSYKD

SEQ ID NO: 6 - protein, 462 aa, Artificial Sequence
  Synthetic
MGSSGMSLAERFSLTLSRSSLVVGRSCVEFEPETVPLLSTLRGKPITFLGLMPPLHEGRREDGEDATVRWLDAQPAKSVVYVALGSEVPLGVEKVHELALGLELAGTRFLWALRKPTGVSDADLLPAGFEERTRGRGVVATRWVPQMSILAHAAVGAFLTHCGWNSTIEGLMFGHPLIMLPIFGDQGPNARLIEAKNAGLQVARNDGDGSFDREGVAAAIRAVAVEEESSKVFQAKAKKLQEIVADMACHERYIDGFIQQLRSYKDDSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDVFHHWAAAAALEHKVPCAMMLLGSAHMIASIADRRLERAETESPAAAGQGRPAAAPTFEVARMKLIRTK

SEQ ID NO: 7 - protein, 459 aa, Hordeum vulgare
MDGNSSSSPLHVVICPWLALGHLLPCLDIAERLASRGHRVSFVSTPRNIARLPPLRPAVAPLVDFVALPLPHVDGLPEGAESTNDVPYDKFELHRKAFDGLAAPFSEFLRAACAEGAGSRPDWLIVDTFHHWAAAAAVENKVPCVMLLLGAATVIAGFARGVSEHAAAAVGKERPAAEAPSFETERRKLMTTQNASGMTVAERYFLTLMRSDLVAIRSCAEWEPESVAALTTLAGKPVVPLGLLPPSPEGGRGVSKEDAAVRWLDAQPAKSVVYVALGSEVPLRAEQVHELALGLELSGARFLWALRKPTDAPDAAVLPPGFEERTRGRGLVVTGWVPQIGVLAHGAVAAFLTHCGWNSTIEGLLFGHPLIMLPISSDQGPNARLMEGRKVGMQVPRDESDGSFRREDVAATVRAVAVEEDGRRVFTANAKKMQEIVADGACHERCIDGFIQQLRSYKA

SEQ ID NO: 8 - protein, 808 aa, Arabidopsis thaliana
MANAERMITRVHSQRERLNETLVSERNEVLALLSRVEAKGKGILQQNQIIAEFEALPEQTRKKLEGGPFFDLLKSTQEAIVLPPWVALAVRPRPGVWEYLRVNLHALVVEELQPAEFLHFKEELVDGVKNGNFTLELDFEPFNASIPRPTLHKYIGNGVDFLNRHLSAKLFHDKESLLPLLKFLRLHSHQGKNLMLSEKIQNLNTLQHTLRKAEEYLAELKSETLYEEFEAKFEEIGLERGWGDNAERVLDMIRLLLDLLEAPDPCTLETFLGRVPMVFNVVILSPHGYFAQDNVLGYPDTGGQVVYILDQVRALEIEMLQRIKQQGLNIKPRILILTRLLPDAVGTTCGERLERVYDSEYCDILRVPFRTEKGIVRKWISRFEVWPYLETYTEDAAVELSKELNGKPDLIIGNYSDGNLVASLLAHKLGVTQCTIAHALEKTKYPDSDIYWKKLDDKYHFSCQFTADIFAMNHTDFIITSTFQEIAGSKETVGQYESHTAFTLPGLYRVVHGIDVFDPKFNIVSPGADMSIYFPYTEEKRRLTKFHSEIEELLYSDVENKEHLCVLKDKKKPILFTMARLDRVKNLSGLVEWYGKNTRLRELANLVVVGGDRRKESKDNEEKAEMKKMYDLIEEYKLNGQFRWISSQMDRVRNGELYRYICDTKGAFVQPALYEAFGLTVVEAMTCGLPTFATCKGGPAEIIVHGKSGFHIDPYHGDQAADTLADFFTKCKEDPSHWDEISKGGLQRIEEKYTWQIYSQRLLTLTGVYGFWKHVSNLDRLEARRYLEMFYALKYRPLAQAVPLAQDD

SEQ ID NO: 9 - protein, 1270 aa, Artificial Sequence
  Synthetic
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSLGSGANAERMITRVHSQRERLNETLVSERNEVLALLSRVEAKGKGILQQNQIIAEFEALPEQTRKKLEGGPFFDLLKSTQEAIVLPPWVALAVRPRPGVWEYLRVNLHALVVEELQPAEFLHFKEELVDGVKNGNFTLELDFEPFNASIPRPTLHKYIGNGVDFLNRHLSAKLFHDKESLLPLLKFLRLHSHQGKNLMLSEKIQNLNTLQHTLRKAEEYLAELKSETLYEEFEAKFEEIGLERGWGDNAERVLDMIRLLLDLLEAPDPCTLETFLGRVPMVFNVVILSPHGYFAQDNVLGYPDTGGQVVYILDQVRALEIEMLQRIKQQGLNIKPRILILTRLLPDAVGTTCGERLERVYDSEYCDILRVPFRTEKGIVRKWISRFEVWPYLETYTEDAAVELSKELNGKPDLIIGNYSDGNLVASLLAHKLGVTQCTIAHALEKTKYPDSDIYWKKLDDKYHFSCQFTADIFAMNHTDFIITSTFQEIAGSKETVGQYESHTAFTLPGLYRVVHGIDVFDPKFNIVSPGADMSIYFPYTEEKRRLTKFHSEIEELLYSDVENKEHLCVLKDKKKPILFTMARLDRVKNLSGLVEWYGKNTRLRELANLVVVGGDRRKESKDNEEKAEMKKMYDLIEEYKLNGQFRWISSQMDRVRNGELYRYICDTKGAFVQPALYEAFGLTVVEAMTCGLPTFATCKGGPAEIIVHGKSGFHIDPYHGDQAADTLADFFTKCKEDPSHWDEISKGGLQRIEEKYTWQIYSQRLLTLTGVYGFWKHVSNLDRLEARRYLEMFYALKYRPLAQAVPLAQDDWT

SEQ ID NO: 10 - protein, 1272 aa, Artificial Sequence
  Synthetic
MDSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDVFHHWAAAAALEHKVPCAMMLLGSAHMIASIADRRLERAETESPAAAGQGRPAAAPTFEVARMKLIRTKGSSGMSLAERFSLTLSRSSLVVGRSCVEFEPETVPLLSTLRGKPITFLGLMPPLHEGRREDGEDATVRWLDAQPAKSVVYVALGSEVPLGVEKVHELALGLELAGTRFLWALRKPTGVSDADLLPAGFEERTRGRGVVATRWVPQMSILAHAAVGAFLTHCGWNSTIEGLMFGHPLIMLPIFGDQGPNARLIEAKNAGLQVARNDGDGSFDREGVAAAIRAVAVEEESSKVFQAKAKKLQEIVADMACHERYIDGFIQQLRSYKDGSGANAERMITRVHSQRERLNETLVSERNEVLALLSRVEAKGKGILQQNQIIAEFEALPEQTRKKLEGGPFFDLLKSTQEAIVLPPWVALAVRPRPGVWEYLRVNLHALVVEELQPAEFLHFKEELVDGVKNGNFTLELDFEPFNASIPRPTLHKYIGNGVDFLNRHLSAKLFHDKESLLPLLKFLRLHSHQGKNLMLSEKIQNLNTLQHTLRKAEEYLAELKSETLYEEFEAKFEEIGLERGWGDNAERVLDMIRLLLDLLEAPDPCTLETFLGRVPMVFNVVILSPHGYFAQDNVLGYPDTGGQVVYILDQVRALEIEMLQRIKQQGLNIKPRILILTRLLPDAVGTTCGERLERVYDSEYCDILRVPFRTEKGIVRKWISRFEVWPYLETYTEDAAVELSKELNGKPDLIIGNYSDGNLVASLLAHKLGVTQCTIAHALEKTKYPDSDIYWKKLDDKYHFSCQFTADIFAMNHTDFIITSTFQEIAGSKETVGQYESHTAFTLPGLYRVVHGIDVFDPKFNIVSPGADMSIYFPYTEEKRRLTKFHSEIEELLYSDVENKEHLCVLKDKKKPILFTMARLDRVKNLSGLVEWYGKNTRLRELANLVVVGGDRRKESKDNEEKAEMKKMYDLIEEYKLNGQFRWISSQMDRVRNGELYRYICDTKGAFVQPALYEAFGLTVVEAMTCGLPTFATCKGGPAEIIVHGKSGFHIDPYHGDQAADTLADFFTKCKEDPSHWDEISKGGLQRIEEKYTWQIYSQRLLTLTGVYGFWKHVSNLDRLEARRYLEMFYALKYRPLAQAVPLAQDD

SEQ ID NO: 11 - protein, 1269 aa, Artificial Sequence
  Synthetic
MDGNSSSSPLHVVICPWLALGHLLPCLDIAERLASRGHRVSFVSTPRNIARLPPLRPAVAPLVDFVALPLPHVDGLPEGAESTNDVPYDKFELHRKAFDGLAAPFSEFLRAACAEGAGSRPDWLIVDTFHHWAAAAAVENKVPCVMLLLGAATVIAGFARGVSEHAAAAVGKERPAAEAPSFETERRKLMTTQNASGMTVAERYFLTLMRSDLVAIRSCAEWEPESVAALTTLAGKPVVPLGLLPPSPEGGRGVSKEDAAVRWLDAQPAKSVVYVALGSEVPLRAEQVHELALGLELSGARFLWALRKPTDAPDAAVLPPGFEERTRGRGLVVTGWVPQIGVLAHGAVAAFLTHCGWNSTIEGLLFGHPLIMLPISSDQGPNARLMEGRKVGMQVPRDESDGSFRREDVAATVRAVAVEEDGRRVFTANAKKMQEIVADGACHERCIDGFIQQLRSYKAGSGANAERMITRVHSQRERLNETLVSERNEVLALLSRVEAKGKGILQQNQIIAEFEALPEQTRKKLEGGPFFDLLKSTQEAIVLPPWVALAVRPRPGVWEYLRVNLHALVVEELQPAEFLHFKEELVDGVKNGNFTLELDFEPFNASIPRPTLHKYIGNGVDFLNRHLSAKLFHDKESLLPLLKFLRLHSHQGKNLMLSEKIQNLNTLQHTLRKAEEYLAELKSETLYEEFEAKFEEIGLERGWGDNAERVLDMIRLLLDLLEAPDPCTLETFLGRVPMVFNVVILSPHGYFAQDNVLGYPDTGGQVVYILDQVRALEIEMLQRIKQQGLNIKPRILILTRLLPDAVGTTCGERLERVYDSEYCDILRVPFRTEKGIVRKWISRFEVWPYLETYTEDAAVELSKELNGKPDLIIGNYSDGNLVASLLAHKLGVTQCTIAHALEKTKYPDSDIYWKKLDDKYHFSCQFTADIFAMNHTDFIITSTFQEIAGSKETVGQYESHTAFTLPGLYRVVHGIDVFDPKFNIVSPGADMSIYFPYTEEKRRLTKFHSEIEELLYSDVENKEHLCVLKDKKKPILFTMARLDRVKNLSGLVEWYGKNTRLRELANLVVVGGDRRKESKDNEEKAEMKKMYDLIEEYKLNGQFRWISSQMDRVRNGELYRYICDTKGAFVQPALYEAFGLTVVEAMTCGLPTFATCKGGPAEIIVHGKSGFHIDPYHGDQAADTLADFFTKCKEDPSHWDEISKGGLQRIEEKYTWQIYSQRLLTLTGVYGFWKHVSNLDRLEARRYLEMFYALKYRPLAQAVPLAQDD

(19) United States
(12) Patent Application Publication
(10) Pub. No.: US 2022/0225651 A1
(43) Pub. Date: Jul. 21, 2022

(54) STEVIOL GLYCOSIDE AND FERULIC ACID FORMULATIONS FOR FOOD AND BEVERAGES

(71) Applicant: Sweegen, Inc., Rancho Santa Margarita, CA (US)

(72) Inventors: Shari B. Mahon, Florence, KY (US); Patricia Hoyle, London (GB)

(73) Assignee: Sweegen, Inc., Rancho Santa Margarita, CA (US)

(21) Appl. No.: 17/575,711

(22) Filed: Jan. 14, 2022

Related U.S. Application Data
(60) Provisional application No. 63/138,759, filed on Jan. 18, 2021.

(51) Int. Cl. A23L 27/30; A23L 2/60; A23K 20/163; A23K 20/111; A23L 29/30; A61K 31/704

(52) U.S. Cl. CPC A23L 27/36; A23L 2/60; A23K 20/163; A23V 2002/00; A23L 29/35; A61K 31/704; A23K 20/111

(57) ABSTRACT

The present invention relates, at least in part to, formulations that have improved caloric profile as well as flavor, taste and/or mouthfeel.

### RELATED APPLICATION

This application claims the benefit under 35 U.S.C. § 119(e) to U.S. Provisional Application No. 63/138,759, filed Jan. 18, 2021, and entitled “STEVIOL GLYCOSIDE AND FERULIC ACID FORMULATIONS FOR FOOD AND BEVERAGES,” the entire contents of each of which are incorporated herein by reference.

### REFERENCE TO SEQUENCE LISTING SUBMITTED AS A TEXT FILE VIA EFS-WEB

The instant application contains a sequence listing which has been submitted in ASCII format via EFS-Web and is hereby incorporated by reference in its entirety. Said ASCII copy, created on Jan. 14, 2022, is named 5210370002U501-SEQ-ZJG and is 68,474 bytes in size.

### FIELD OF THE INVENTION

The present invention relates, at least in part to, low or non-caloric sweetener compositions based on steviol glycosides, such as Reb E and Reb I, combined with ferulic acid, as well as compositions thereof, such as oral products, consumables, beverages and pharmaceuticals. More specifically it relates to particular formulations of rebaudiosides and ferulic acid that can elicit a pleasant oral sensory experience.

### SUMMARY OF THE INVENTION

The present invention relates to formulations with rebaudiosides and ferulic acid that have beneficial properties. The formulations can modify the taste perception of orally consumable products (such as that they can exhibit enhanced sweetness, improved onset of sweetness, improved time and intensity of sweetness, and/or mask bitterness and/or off notes). The formulations comprise Rebaudioside E (Reb E) and Rebaudioside I (Reb I) in varying quantities, as well as ferulic acid, and can exhibit taste characteristics similar to sugar sweetener systems in carbonated (Cola and Non-Cola) and non-carbonated beverages and concentrates, protein-based products, liquid dairy, yogurt, condiments, baked goods, jams, jellies, and spreads. Additionally, the formulations can provide for higher solubility than the individual use of Reb E and Reb I with a taste profile closer to sucrose than individual rebaudiosides, specifically, Reb E or Reb I alone, in some embodiments. Further, the ferulic acid can act as an antioxidant, stabilizing the rebaudiosides with which it is formulated, in some embodiments.

Some aspects provide compositions (e.g., formulations) comprising Reb E, Reb I, and ferulic acid. In one aspect, Reb E is present in the amount of from about 3 ppm to about 45 ppm. In an embodiment of any one of the methods or compositions provided herein, Reb E is present in the amount of from about 5 ppm to about 40 ppm, from about 10 ppm to about 35 ppm, from about 15 ppm to about 30 ppm, from about 20 ppm to about 25 ppm, from about 3 ppm to about 40 ppm, from about 3 ppm to about 35 ppm, from about 3 ppm to about 30 ppm, from about 3 ppm to about 25 ppm, from about 3 ppm to about 20 ppm, from about 3 ppm to about 15 ppm, from about 3 ppm to about 10 ppm, from about 5 ppm to about 45 ppm, from about 10 ppm to about 45 ppm, from about 15 ppm to about 45 ppm, from about 20 ppm to about 45 ppm, from about 25 ppm to about 45 ppm, from about 30 ppm to about 45 ppm or from about 35 ppm to about 45 ppm.

In another aspect, Reb I is present in the amount of from about 2 ppm to about 30 ppm. In an embodiment of any one of the methods or compositions provided herein, Reb I is present in the amount of from about 5 ppm to about 25 ppm, from about 10 ppm to about 20 ppm, from about 2 ppm to about 25 ppm, from about 2 ppm to about 20 ppm, from about 2 ppm to about 15 ppm, from about 2 ppm to about 10 ppm, from about 5 ppm to about 30 ppm, from about 10 ppm to about 30 ppm, from about 15 ppm to about 30 ppm or from about 20 ppm to about 30 ppm.

In another aspect, ferulic acid is present in the amount of from about 0.8 ppm to about 30 ppm. In another aspect, ferulic acid is present in the amount of from about 0.8 ppm to about 12 ppm. In an embodiment of any one of the methods or compositions provided herein, ferulic is present in the amount of from about 1 ppm to about 30 ppm, from about 5 ppm to about 25 ppm, from about 10 ppm to about 20 ppm, from about 1 ppm to about 25 ppm, from about 1 ppm to about 20 ppm, from about 1 ppm to about 15 ppm, from about 1 ppm to about 10 ppm, from about 5 ppm to about 30 ppm, from about 5 ppm to about 25 ppm, from about 5 ppm to about 20 ppm, from about 5 ppm to about 15 ppm or from about 5 ppm to about 10 ppm.

In some embodiments, Reb E is present in the composition in an amount of from about 3 ppm to about 45 ppm (e.g., about 3 ppm to about 45 ppm, about 5 ppm to about 40 ppm, about 10 ppm to about 30 ppm, about 15 ppm to about 25 ppm), Reb I is present in the composition in an amount of from about 2 ppm to about 30 ppm (e.g., about 2 ppm to about 30 ppm, about 5 ppm to about 25 ppm, about 10 ppm to about 20 ppm), and/or wherein ferulic acid is present in the composition in an amount of about 0.8 to about 30 ppm (e.g., about 0.8 to about 12 ppm, about 1 to about 15 ppm, about 5 to about 20 ppm, or about 10 to about 15 ppm).

In another aspect, the composition (e.g., formulation) comprises 45 ppm Reb E, 30 ppm Reb I and 12 ppm ferulic acid.

In another aspect, the composition (e.g., formulation) comprises about 40-60 wt. % Reb E, about 25-45 wt. % Reb I and about 5-25 wt. % ferulic acid, all percentages being on an anhydrous wt./wt. basis. In an embodiment of any one of the methods or compositions provided herein, the composition (e.g., formulation) comprises about 40-55 wt. %, 40-50 wt.

%, 40-45 wt. %, 45-60 wt. %, 50-60 wt. % or 55-60 wt. % Reb E; about 25-45 wt. %, 25-40 wt. %, 25-35 wt. %, 25-30 wt. %, 30-45 wt. %, 35-45 wt. % or 40-45 wt. % Reb I; and about 5-25 wt. %, 5-20 wt. %, 5-15 wt. %, 5-10 wt. %, 10-25 wt. %, 15-25 wt. % or 20-25 wt. % ferulic acid, all percentages being on an anhydrous wt./wt. basis. In one embodiment of any one of the methods or compositions provided herein, the amounts of Reb E, Reb I and ferulic acid is based on their amounts relative to all actives on a dry basis. In one embodiment of any one of the methods or compositions provided herein, the amounts of Reb E, Reb I and ferulic acid is based on their amounts relative to all actives (e.g., not counting flavor solvents) on a dry basis.

In some embodiments, a composition provided herein is a composition described in the Examples.

In an embodiment of any one of the compositions or methods provided herein, the composition (e.g., formulation) comprise purified steviol glycosides, such as Reb E and Reb I, and ferulic acid.

In some embodiments, the composition (e.g., formulation) is present in a consumable, where Reb E is present in a concentration of about 3-45 ppm (e.g., about 3 ppm to about 45 ppm, about 5 ppm to about 40 ppm, about 10 ppm to about 30 ppm, about 15 ppm to about 25 ppm), Reb I is present in a concentration of about 2-30 ppm (e.g., about 2 ppm to about 30 ppm, about 5 ppm to about 25 ppm, about 10 ppm to about 20 ppm), and ferulic acid is present in a concentration of about 0.8-12 ppm e.g., about 0.8 to about 12 ppm, about 1 to about 15 ppm, about 5 to about 20 ppm, or about 10 to about 15 ppm).

In some embodiments, the consumable comprises about 0.2-0.30 wt. % the composition, on an anhydrous wt./wt. basis.

Oral compositions, pharmaceutical compositions, beverages, food compositions, amino feed products, powders or liquids comprising a composition described herein are provided. In some embodiments, a liquid comprising a composition described herein further comprises a solvent. In some embodiments, the solvent is a liquid, food grade and/or organic solvent. In some embodiments, the solvent is ethyl alcohol, propylene glycol, glycerin, SM00056, or water and/or combinations thereof. In some embodiments, the solvent is propylene glycol or SM00056. In some embodiments, a powder comprising a composition described herein further comprises dextrin or maltodextrin. In some embodiments, a pharmaceutical composition comprising a composition described herein further comprises a pharmaceutically acceptable excipient

Also provided herein are methods for optimizing orally consumable compositions with the formulations provided to optimize health improvements in end consumers, such as in the form of a food item with a less dense calorie profile while retaining a desirable taste profile. This is also provided for companion animals that may benefit from a calorie reduction in their daily diets. For animals produced for market the formulations provided can be used for changing the taste profile of lower quality feed or enhancing the flavor of feed containing nutrients that may be needed but that have an enhanced bitterness or off-flavor.

In an embodiment, the formulations of the invention can allow for up to 100% sugar reduction in a wide-range of food and beverage products with higher solubility characteristics than from other individual rebaudiosides and known blends, while maintaining the desired flavor and taste profile and providing an onset of sweetness that is almost identical to that of table sugar.

In an embodiment, the formulations can improve the caloric profile of food and beverages for the elderly and the unwell, relative to the nutrients provided. In nutrition drinks designed for weight loss and/or nutrient delivery the taste characteristics provided with the formulations can be very similar to table sugar allowing for up to 100% sugar reduction in food and beverage products while improving overall solubility in higher concentrations and masking enhanced bitterness or off-flavors.

Also provided in an aspect are commercially valuable processes for producing low-calorie composite sweetener compositions comprising the formulations provided herein for use in various food/feed products and beverages.

In an embodiment the formulations of the invention may be produced by genetically modified microbes designed to produce sufficient quantities of otherwise rare, expensive or difficult to extract steviol glycosides. It is apparent that this may be done with a much more limited geographic footprint than that needed for the production/breeding of the Stevia rebaudiana plant.

In some embodiments, at least one rebaudioside and/or ferulic acid is made by a genetically modified microbe. In some embodiments, the genetically modified microbe comprises one or more proteins having UDP-glycosyltransferase (UGT) and/or sucrose synthase (SUS) activity. Other methods, including extraction and synthetic methods can also be used for producing the at least one rebaudioside and/or ferulic acid.

The present disclosure provides, in some aspects, methods for creating or enhancing the sweetness of any one of the compositions provided herein, comprising adding an amount of a formulation to produce the desired degree of sweetness to a composition. The composition may be for consumption of a food, beverage or pharmaceutical composition in some embodiments.

Other features and advantages of the present disclosure will become apparent in the following detailed description of preferred embodiments of the disclosure.

### DETAILED DESCRIPTION

Before describing the present invention in detail, it is to be understood that this invention is not limited to particularly exemplified materials or process parameters as such may, of course, vary. It is also to be understood that the terminology used herein is for the purpose of describing particular embodiments of the invention only, and is not intended to be limiting of the use of alternative terminology to describe the present invention.

All publications, patents and patent applications cited herein, whether supra or infra, are hereby incorporated by reference in their entirety for all purposes.

As used herein, the term “comprise” or variations thereof such as “comprises” or “comprising” are to be read to indicate the inclusion of any recited integer (e.g. a feature, element, characteristic, property, method/process step or limitation) or group of integers (e.g. features, elements, characteristics, properties, method/process steps or limitations) but not the exclusion of any other integer or group of integers. Thus, as used herein, the term “comprising” is inclusive and does not exclude additional, unrecited integers or method/process steps.

In embodiments of any one of the compositions and methods provided herein, “comprising” may be replaced with “consisting essentially of” or “consisting of”. The phrase “consisting essentially of” is used herein to require the specified integer(s) or steps as well as those which do not materially affect the character or function of the claimed invention. As used herein, the term “consisting” is used to indicate the presence of the recited integer (e.g. a feature, element, characteristic, property, method/process step or limitation) or group of integers (e.g. features, elements, characteristics, properties, method/process steps or limitations) alone.

### DEFINITIONS

Unless defined otherwise, all technical and scientific terms used herein have the same meaning as commonly understood by one of ordinary skill in the art to which the disclosure belongs. Although any methods and materials similar to or equivalent to those described herein may be used in the practice or testing of the present disclosure, the preferred materials and methods are described below.

As used herein, “synthetic” or “organically synthesized” or “chemically synthesized” or “organically synthesizing” or “chemically synthesizing” or “organic synthesis” or “chemical synthesis” are used to refer to preparing the compounds through a series of chemical reactions; this does not include extracting the compound, for example, from a natural source.

The term “orally consumable product” as used herein refers to any beverage, food product, dietary supplement, nutraceutical, pharmaceutical composition, dental hygienic composition and cosmetic product which are contacted with the mouth of man or animal, including substances that are taken into and subsequently ejected from the mouth and substances which are drunk, eaten, swallowed, or otherwise ingested; and that are considered safe for human or animal consumption when used in a generally acceptable range of concentrations.

The term “food product” or “food composition” as used herein includes fruits, vegetables, juices, meat products such as ham, bacon and sausage; egg products, fruit concentrates, gelatins and gelatin-like products such as jams, jellies, preserves, and the like; milk products such as ice cream, sour cream, yogurt, and sherbet; icings, syrups including molasses; corn, wheat, rye, soybean, oat, rice and barley products, cereal products, nut meats and nut products, cakes, cookies, confectionaries such as candies, gums, fruit flavored drops, and chocolates, chewing gum, mints, creams, icing, ice cream, pies and breads. “Food product” also refers to condiments such as herbs, spices and seasonings, flavor enhancers, such as monosodium glutamate. “Food product” further also includes prepared packaged products, such as dietetic sweeteners, liquid sweeteners, tabletop flavorings, granulated flavor mixes which upon reconstitution with water provide non-carbonated drinks, instant pudding mixes, instant coffee and tea, coffee whiteners, malted milk mixes, pet foods, livestock feed, tobacco, and materials for baking applications, such as powdered baking mixes for the preparation of breads, cookies, cakes, pancakes, donuts and the like. “Food product” also includes diet or low-calorie food and beverages containing little or no sucrose.

As used herein, the term “sweetness intensity” refers to the relative strength of sweet sensation as can be observed or experienced by an individual, e.g., a human, or a degree or amount of sweetness detected by a taster, for example on a Brix scale.

As used herein, the term “enhancing the sweetness” refers to the effect of a sweetener in increasing, augmenting, intensifying, accentuating, magnifying, and/or potentiating the sensory perception of one or more sweetness characteristics of an orally consumable product as provided herein as compared to a corresponding orally consumable product that does not contain the sweetener.

As used herein, the term “off-taste(s)” refers to an amount or degree of taste that is not characteristically or usually found or expected in an orally consumable product. For example, an off-taste is an undesirable taste of a sweetened consumable, such as, a bitter taste, a licorice-like taste, a metallic taste, an aversive taste, an astringent taste, a delayed sweetness onset, a lingering sweet aftertaste, and the like, etc.

As used herein, the term “wt. %” refers to the weight % of a compound (e.g., a Rebaudioside) relative to the total weight of all compounds in a composition.

As used herein, the term “ppm” refers to part(s) per million by weight, for example, the weight of a compound, such as a Rebaudioside (in milligrams) per kilogram, of a composition, such as an orally consumable product, containing such compound (i.e., mg/kg) or the weight of a compound, such as a Rebaudioside (in milligrams) per liter, of a composition, such as an orally consumable product, containing such compound (i.e., mg/L); or by volume, for example the volume of a compound, such as a Rebaudioside (in milliliters) per liter, of a composition, such as an orally consumable product containing such compound (i.e., ml/L).

As used herein, the term “carbohydrate sweetener” includes caloric sweeteners, such as, sucrose, fructose, glucose, high fructose corn syrup (containing fructose and glucose), xylose, arabinose, rhamnose, and sugar alcohols, such as erythritol, xylitol, mannitol, sorbitol, and inositol.

As used herein, the term “flavoring” or the like refers to any food-grade material that may be added to or present in an orally consumable product to provide a desired flavor.

The term “isolated” is used according to its ordinary and customary meaning as understood by a person of ordinary skill in the art, and when used in the context of an isolated nucleic acid or an isolated polypeptide, is used without limitation to refer to a nucleic acid or polypeptide that, by the hand of man, exists apart from its native environment and is therefore not a product of nature. An isolated nucleic acid or polypeptide can exist in a purified form or can exist in a non-native environment such as, for example, in a transgenic host cell.

The terms “recombinant,” “heterologous,” and “exogenous,” when used herein in connection with polynucleotides, are used according to their ordinary and customary meanings as understood by a person of ordinary skill in the art, and are used without limitation to refer to a polynucleotide (e.g., a DNA sequence or a gene) that originates from a source foreign to the particular host cell or, if from the same source, is modified from its original form. Thus, a heterologous gene in a host cell includes a gene that is endogenous to the particular host cell but has been modified through, for example, the use of site-directed mutagenesis or other recombinant techniques. The terms also include non-naturally occurring multiple copies of a naturally occurring DNA sequence. Thus, the terms refer to a DNA segment that is foreign or heterologous to the cell, or homologous to the cell but in a position or form within the host cell in which the element is not ordinarily found.

Similarly, the terms “recombinant,” “heterologous,” and “exogenous,” when used herein in connection with a polypeptide or amino acid sequence, means a polypeptide or amino acid sequence that originates from a source foreign to the particular host cell or, if from the same source, is modified from its original form. Thus, recombinant DNA segments can be expressed in a host cell to produce a recombinant polypeptide.

As used herein, the singular form “a”, “an”, and “the” includes plural references unless indicated otherwise.

Reference to “about” a value or parameter herein refers to the usual error range for the respective value readily known to the skilled person in this technical field. Reference to “about” for a value or parameter herein includes (and describes) aspects that are directed to that value or parameter per se. For example, description referring to “about X” includes description of “X.” In an embodiment of any one of the compositions or methods provided herein, any amount can recited herein can refer to the amount alone and without “about”.

### Introduction to the Steviol Glycoside and Ferulic Acid Formulations

Many studies have focused on the connection of sugar consumption with obesity and other pathologies such as diabetes. Consumers and food companies alike are interested in calorie reduction through the use of sugar alternatives. There is also significant interest in the reduction of calories for companion animals or the use of sweeteners to make certain feed products more palatable.

Non-caloric natural and synthetic high-potency sweeteners are known, but they often possess flavor profiles that are not as desirable to consumers as natural caloric sweeteners. Thus, it is desirable to develop improved non-caloric sweeteners that can be substituted for sugar and that have a more desirable taste profile. The species Stevia rebaudiana (“Stevia”) is the source of certain naturally occurring sweet steviol glycosides. Considerable research and development has focused on the use of sweet steviol glycosides of Stevia as non-caloric sweeteners, but each of the various steviol glycosides have their limitations in terms of taste, solubility or off-flavor. Surprisingly, formulations with beneficial properties (such as improved solubility, flavor (e.g., masked bitterness), caloric profile, etc.) have been discovered.

In an embodiment, the formulations provided can provide the “full” sweetness needed for many food and beverage applications. This typically is difficult to achieve with a single rebaudioside given the off-flavors which may attend to many of them, such as for example Reb A, due to its perceptible bitterness at concentrations above about 200 ppm. More specifically, the formulations provided can provide high intensity sweeteners that may be added to the food product, beverage, or other consumables, such an oral or pharmaceutical composition. The compositions provided, in an embodiment, can comprise about 50 to about 800 ppm of steviol glycosides without the composition having an unacceptable bitterness. Similarly, the bitterness in a composition can be masked but provide the desired sweetness.

While in general the formulations provided herein can be used for lower or non-caloric purposes, there may be instances where when used with other compounds such as protein or protein sweetener the result may be an increase in the calorific value per unit sweetness of a composition as provided herein.

### Methods of Production

Steviol glycosides can be isolated from Stevia rebaudiana leaves. Steviol glycosides are used as high intensity, low-calorie sweeteners and are significantly sweeter than sucrose. As natural sweeteners, different steviol glycosides have different degrees of sweetness and after-taste. For example, stevioside is 100-150 times sweeter than sucrose with bitter after-taste. Rebaudioside C is between 40-60 times sweeter than sucrose. Dulcoside A is about 30 times sweeter than sucrose. Steviol glycosides with known structures include Rebaudioside E and Rebaudioside I.

| | | | |
|---|---|---|---|
| Rebaudioside E | | C₄₄H₇₀O₂₃ | 966 |
| Rebaudioside I | 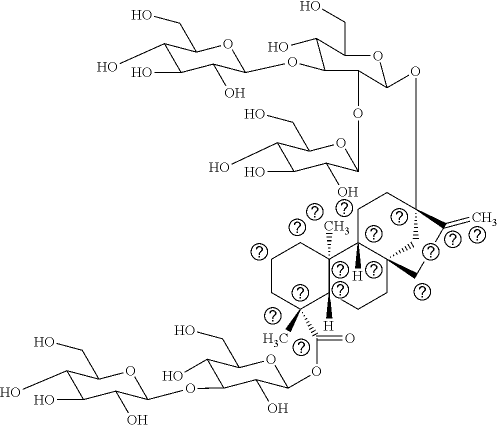 | C₅₀H₈₀O₂₈ | 1129.15 |

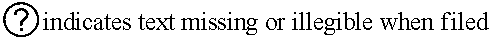

The majority of steviol glycosides are formed by several glycosylation reactions of steviol, which are typically catalyzed by the UDP-glycosyltransferases (UGTs) using uridine 5′-diphosphoglucose (UDP-glucose) as a donor of the sugar moiety. UGTs in plants make up a very diverse group of enzymes that transfer a glucose residue from UDP-glucose to steviol. For example, glycosylation of the C-2′ of the 19-O-glucose of the stevioside yields Rebaudioside E.

Any suitable technique known in the art for isolating and/or purifying compounds, such as Rebaudiosides from plants, such as Stevia, may be used. For example, Rebaudiosides can be isolated and/or purified from Stevia plant material utilizing one or more of the techniques described in U.S. Pat. Nos. 3,723,410;4,082,858; 4,361,697; 4,599,403; 5,112,610; 5,962,678; 8,299,224; 8,414,951; U.S. Patent Application Publication Nos. 2006/0083838; 2006/0134292; 2007/0082103; 2008/0300402; and Chaturvedula, VSP and Prakash, I, Eur. Chem. Bull. 2013, 2(5), 298-302. Such techniques are incorporated herein by reference. Alternatively, the compounds can be recombinantly produced or chemically synthesized using methods well known to those of skill in the art.

In some embodiments, glycosides from leaves, such as Rebaudiosides, can be extracted using either water or organic solvent extraction. Supercritical fluid extraction and steam distillation can also be used. In other embodiments, Rebaudiosides can be recovered from Stevia plants using membrane technology. In some embodiments, production of an extract typically includes extraction of plant material with water or an water-organic solvent mixture, precipitation of high molecular weight substances, deionization and decolorization, purification on specific macroporous polymeric adsorbents, concentration, and drying.

In other embodiments, extracts of Stevia leaves may be purified to concentrate a selected component of the Stevia extract. For example, column chromatography may be used to isolate Rebaudiosides from the other diterpene glycosides. In some embodiments, following chromatographic separation, the produced Rebaudioside may optionally be recrystallized at least once, or at least twice, or at least three times, to obtain a Stevia extract containing a desired level of purity of the Rebaudioside.

In some embodiments, an extract used in the formulations provided herein has a purity of about 50% to about 100% by weight, about 55% to about 100% by weight, about 60% to about 100% by weight, about 65% to about 100% by weight, about 70% to about 100% by weight, about 75% to about 100% by weight, about 80% to about 100% by weight, about 85% to about 100% by weight, about 86% to about 100% by weight, about 87% to about 100% by weight, about 88% to about 100% by weight, about 89% to about 100% by weight, about 90% to about 100% by weight, about 91% to about 100% by weight, about 92% to about 100% by weight, about 93% to about 100% by weight, about 94% to about 100% by weight, about 95% to about 100% by weight, about 96% to about 100% by weight, about 97% to about 100% by weight, about 98% to about 100% by weight, or about 99% to about 100% by weight.

Alternatively, an extract used in the formulations provided herein has a purity of about 50% to about 100% by weight, about 50% to about 99% by weight, about 50% to about 98% by weight, about 50% to about 97% by weight, about 50% to about 96% by weight, about 50% to about 95% by weight, about 50% to about 94% by weight, about 50% to about 93% by weight, about 50% to about 92% by weight, about 50% to about 91% by weight, about 50% to about 90% by weight, about 50% to about 85% by weight, about 50% to about 80% by weight, about 50% to about 75% by weight, about 50% to about 70% by weight, about 50% to about 65% by weight, about 50% to about 60% by weight, or about 50% to about 55% by weight. For example, an extract used in a formulation provided herein may have a purity of about 50%, about 55%, about 60%, about 65%, about 70%, about 75%, about 80%, about 85%, about 86%, about 87%, about 88%, about 89%, about 90%, about 91%, about 92%, about 93%, about 94%, about 95%, about 96%, about 97%, about 98%, about 99%, or about 100% by weight, including any range in between these values.

The purity of Rebaudiosides, such as those extracted, isolated, and/or purified from Stevia plants, can be assayed using any suitable method known in the art. For example, chromatography, such as HPLC, may be used to test the purity of Rebaudioside extracts.

In some embodiments, production can be accomplished through the utilization of microbial strains to produce Rebaudiosides in high yield and purity to allow commercial incorporation into orally consumable products (See, e.g., U.S. Pat. Nos. 9,988,414, 9,522,929, 10,010,099, 10,010,101, 10,081,826, 10,253,344 all of which, including the methods of production, are incorporated herein by reference).

In some embodiments, Rebaudiosides may be produced by recombinantly expressing enzymes in a microbial system. In some embodiments, the host cell is selected from the group consisting of Escherichia; Salmonella; Bacillus; Acinetobacter; Streptomyces; Corynebacterium; Methylosinus; Methylomonas; Rhodococcus; Pseudomonas; Rhodobacter; Synechocystis; Saccharomyces; Zygosaccharomyces; Kluyveromyces; Candida; Hansenula; Debaryomyces; Mucor; Pichia; Torulopsis; Aspergillus; Arthrobotlys; Brevibacteria; Microbacterium; Arthrobacter; Citrobacter; Klebsiella; Pantoea; Corynebacterium; Clostridium (e.g., Clostridium acetobutylicum). In some embodiments, the host cell is a cell isolated from plants selected from the group consisting of soybean; rapeseed; sunflower; cotton; corn; tobacco; alfalfa; wheat; barley; oats; sorghum; rice; broccoli; cauliflower; cabbage; parsnips; melons; carrots; celery; parsley; tomatoes; potatoes; strawberries; peanuts; grapes; grass seed crops; sugar beets; sugar cane; beans; peas; rye; flax; hardwood trees; softwood trees; forage grasses; Arabidopsis thaliana; rice (Oryza sativa); Hordeum yulgare; switchgrass (Panicum vigratum); Brachypodium spp.; Brassica spp.; and Crambe abyssinica. In some embodiments, the cell is a bacterial cell, such as E. coli, or a yeast cell, such as a Saccharomyces cell, Pichia cell, or a Yarrowia cell. In some embodiments, the cell is an algal cell or a plant cell.

In some embodiments, Rebaudiosides of the formulations provided herein are produced in a reaction mixture including a start compound (e.g., any natural or synthetic compound capable of being converted into a steviol glycoside compound in a reaction catalyzed by one or more enzymes); a substrate selected from the group consisting of sucrose, uridine diphosphate (UDP) and uridine diphosphate-glucose (UDP-glucose); and one or more enzymes, such as a UDP-glycosyltransferase. Suitable UDP-glycosyltransferases for producing Rebaudiosides in either a microbial system or an in vitro reaction mixture include any UGT known in the art as capable of catalyzing one or more reactions in the biosynthesis of steviol glycoside compounds, such as, without limitation, EUGT11 (GenBank Accession No. AC133334), HV1 (GenBank Accession No. BAJ98242.1), UGT76G1 (Genbank Accession No. AAR06912.1), UGT85C2 (GenBank Accession No. AAR06916.1), UGT74G1 (GenBank Accession No. AAR06920.1), or the functional homologs, fragments, or variants thereof.

In some embodiments, the UDP-glycotransferase used in any one of the methods described herein is EUGT11, or any functional fragments or variants thereof. EUGT11 is a UGT having 1,2-19-O-glucose and 1,2-13-O-glucose glycosylation activity. EUGT11 is known to catalyze the production of stevioside to Rebaudioside E. EUGT11 also has 1,2-19-O-glucose glycosylation activity. In some embodiments, the UDP-glycotransferase used in any one of the methods described herein is HV1, or any functional fragments or variants thereof. HV1 is a UGT with a 1,2-19-O-glucose glycosylation activity that can produce related steviol glycosides, such as Rebaudioside E. HV1 also can convert Reb KA to Reb E.

In some embodiments, the reaction mixture further comprises additional enzymes (e.g., sucrose synthase or SUS) to improve the efficiency or modify the outcome of the overall biosynthesis of steviol glycoside compounds. For example, the additional enzyme may regenerate the UDP-glucose needed for the glycosylation reaction by converting the UDP produced from the glycosylation reaction back to UDP-glucose (using, for example, sucrose as a donor of the glucose residue), thus improving the efficiency of the glycosylation reaction.

Standard recombinant DNA and molecular cloning techniques used here are well known in the art and are described, for example, by Sambrook, J., Fritsch, E. F. and Maniatis, T. MOLECULAR CLONING: A LABORATORY MANUAL, 2nd ed.; Cold Spring Harbor Laboratory: Cold Spring Harbor, N.Y., 1989 (hereinafter “Maniatis”); and by Silhavy, T. J., Bennan, M. L. and Enquist, L. W. EXPERIMENTS WITH GENE FUSIONS; Cold Spring Harbor Laboratory: Cold Spring Harbor, N.Y., 1984; and by Ausubel, F. M. et al., IN CURRENT PROTOCOLS IN MOLECULAR BIOLOGY, published by Greene Publishing and Wiley-Interscience, 1987; (the entirety of each of which is hereby incorporated herein by reference).

### Rebaudioside E

Rebaudioside E is a steviol glycoside produced in Stevia plants. Rebaudioside E has the molecular formula C₄₄H₇₀O₂₃ and the IUPAC name, [(2S,3R,4S,5S,6R)-4,5-dihydroxy-6-(hydroxymethyl)-3-[(2S,3R,4S,5S,6R)-3,4,5-trihydroxy-6-(hydroxymethyl)oxan-2-yl]oxyoxan-2-yl] (1R,4S,5R,9S,10R,13S)-13-[(2S,3R,4S,5S,6R)-4,5-dihydroxy-6-(hydroxymethyl)-3-[2S,3R,4S,5S,6R)-3,4,5-trihydroxy-6-(hydroxymethyl)oxan-2-yl]oxyoxan-2-yl]oxy-5,9-dimethyl-14-methylidenetetracyclo[11.2.1.0^{1.10}.0^{4.9}]hexadecane-5-carboxylate.

Rebaudioside E may be purified from Stevia leaf extracts, or recombinantly or synthetically produced. In some embodiments, Rebaudioside E is produced via covalently coupling one or more glucoses to stevioside, rubusoside, or rebaudioside KA by an UDP-glycosyltransferase selected from the group consisting of HV1, EUGT11, UGT76G1, a HV1-SUS fusion enzyme, a EUGT11-SUS fusion enzyme, and a UTG76G1-SUS fusion enzyme.

In some embodiments, Rebaudioside E is produced via covalently coupling a glucose to rebaudioside KA by the HV1, EUGT11, HV1-SUS fusion enzyme, or EUGT11-SUS fusion enzyme. In some embodiments, Rebaudioside E is produced from a reaction mixture comprising rebaudioside KA, substrates selected from the group consisting of sucrose, uridine diphosphate (UDP), uridine diphosphate-glucose (UDP-glucose), and combinations thereof, and EUGT11 (e.g., SEQ ID NO: 5), HV1 (e.g., SEQ ID NO: 7), EUGT11-SUS fusion enzyme (e.g., SEQ ID NO: 10), or HV1-SUS fusion enzyme (e.g., SEQ ID NO: 11), with or without additional sucrose synthase (e.g., SEQ ID NO: 8).

In some embodiments, Rebaudioside E is produced via covalently coupling a glucose to stevioside by the HV1, EUGT11, HV1-SUS fusion enzyme, or EUGT11-SUS fusion enzyme. In some embodiments, Rebaudioside E is produced from a reaction mixture comprising stevioside, substrates selected from the group consisting of sucrose, uridine diphosphate (UDP), uridine diphosphate-glucose (UDP-glucose), and combinations thereof, and EUGT11 (e.g., SEQ ID NO: 5), HV1 (e.g., SEQ ID NO: 7), EUGT11-SUS fusion enzyme (e.g., SEQ ID NO: 10), or HV1-SUS fusion enzyme (e.g., SEQ ID NO: 11), with or without additional sucrose synthase (e.g., SEQ ID NO: 8).

In some embodiments, Rebaudioside E is produced via covalently coupling two glucoses to rubusoside by the HV1, EUGT11, HV1-SUS fusion enzyme, or EUGT11-SUS fusion enzyme. For example, a glucose is covalently coupled to the rubusoside to produce Rebaudioside KA. A glucose can then be covalently coupled to the Rebaudioside KA to produce Rebaudioside E. In some embodiments, Rebaudioside E is produced from a reaction mixture comprising rubusoside, substrates selected from the group consisting of sucrose, uridine diphosphate (UDP), uridine diphosphate-glucose (UDP-glucose), and combinations thereof, and EUGT11 (e.g., SEQ ID NO: 5), HV1 (e.g., SEQ ID NO: 7), EUGT11-SUS fusion enzyme (e.g., SEQ ID NO: 10), or HV1-SUS fusion enzyme (e.g., SEQ ID NO: 11), with or without additional sucrose synthase (e.g., SEQ ID NO: 8).

### Rebaudioside I

Rebaudioside I has the molecular formula C₅₀H₈₀O₂₈ and the IUPAC name, 13-[(2-O-β-D-glucopyranosyl-3-O-β-D-glucopyranosyl)-β-D-glucopyranosyl)oxy]-ent-kaur-16-en-19oic acid-(3-O-β-D-glucopyranosyl)-β-D-glucopyranosyl), ester.

In some embodiments, Rebaudioside I is produced via covalently coupling a glucose to a steviol glycoside (e.g., rebaudioside A) by an UGT76G1, a UTG76G1-SUS fusion enzyme, or UGT76G1 variants such as UGT76G1 CP1, UGT76G1 CP2, and UGT76G1 L200A. In some embodiments, Rebaudioside I produced by a reaction mixture comprising a steviol glycoside (e.g., rebaudioside A); a substrate selected from the group consisting of sucrose, uridine diphosphate (UDP), and uridine diphosphate-glucose (UDP-glucose); and UGT76G1 (e.g., SEQ ID No: 1), UGT76G1-SUS fusion enzyme (e.g., SEQ ID NO: 9), UTG76G1 CP1 variant (e.g., SEQ ID NO: 3), UTG76G1 CP2 variant (e.g., SEQ ID NO: 4), or UTG76G1 L200A variant (e.g., SEQ ID NO: 2), with or without additional sucrose synthase (e.g., SEQ ID NO: 8).

### Examples of Enzymes Used for Synthesizing Rebaudiosides

| Name | Sequences |
|---|---|
| UGT76G1 | MENKTETTVRRRRRIILFPVPFQGHINPILQLANVLYSKGFSITIFHTNFNKPKTSNYPHFTFRFILDNDPQDERISNLP |
| WT Amino | THGPLAGMRIPIINEHGADELRRELELLMLASEEDEEVSCLITDALWYFAQSVADSLNLRRLVLMTSSLFNFHAHVS |
| Acid | LPQFDELGYLDPDDKTRLEEQASGFPMLKVKDIKSAYSNWQILKEILGKMIKQTKASSGVIWNSFKELEESELETVI |
| | REIPAPSFLIPLPKHLTASSSSLLDHDRTVFQWLDQQPPSSVLYVSFGSTSEVDEKDFLEIARGLVDSKQSFLWVVRP |
| | GFVKGSTWVEPLPDGFLGERGRIVKWVPQQEVLAHGAIGAFWTHSGWNSTLESVCEGVPMIFSDFGLDQPLNARY |
| | MSDVLKVGVYLENGWERGEIANAIRRVMVDEEGEYIRQNARVLKQKADVSLMKGGSSYESLESLVSYISSL (SEQ |
| | ID NO: 1) |
| UGT76G1 | MENKTETTVRRRRRIILFPVPFQGHINPILQLANVLYSKGFSITIFHTNFNKPKTSNYPHFTFRFILDNDPQDERISNLP |
| L200A | THGPLAGMRIPIINEHGADELRRELELLMLASEEDEEVSCLITDALWYFAQSVADSLNLRRLVLMTSSLFNFHAHVS |
| Amino | LPQFDELGYLDPDDKTRLEEQASGFPMLKVKDIKSAYSNWQIAKEILGKMIKQTKASSGVIWNSFKELEESELETVI |
| Acid | REIPAPSFLIPLPKHLTASSSSLLDHDRTVFQWLDQQPPSSVLYVSFGSTSEVDEKDFLEIARGLVDSKQSFLWVVRP |
| | GFVKGSTWVEPLPDGFLGERGRIVKWVPQQEVLAHGAIGAFWTHSGWNSTLESVCEGVPMIFSDFGLDQPLNARY |
| | MSDVLKVGVYLENGWERGEIANAIRRVMVDEEGEYIRQNARVEKQKADVSLMKGGSSYESLESLVSYISSL (SEQ |
| | ID NO: 2) |
| UGT76G1 | MNWQILKEILGKMIKQTKASSGVIWNSFKELEESELETVIREIPAPSFLIPLPKHLTASSSSLLDHDRTVFQWLDQQP |
| CP1 | PSSVLYVSFGSTSEVDEKDFLEIARGLVDSKQSFLWVVRPGFVKGSTWVEPLPDGFLGERGRIVKWVPQQEVLAH |
| Amino | GAIGAFWTHSGWNSTLESVCEGVPMIFSDFGLDQPLNARYMSDVEKVGVYLENGWERGEIANAIRRVMVDEEGE |
| Acid | YIRQNARVLKQKADVSLMKGGSSYESLESLVSYISSLENKTETTVRRRRRIILFPVPFQGHINPILQLANVLYSKGFSI |
| | TIFHTNFNKPKTSNYPHFTFRFILDNDPQDERISNLPTHGPLAGMRIPIINEHGADELRRELELLMLASEEDEEVSCLI |
| | TDALWYFAQSVADSLNLRRLVLMTSSLFNFHAHVSLPQFDELGYLDPDDKTRLEEQASGFPMEKVKDIKSAYS |
| | (SEQ ID NO: 3) |
| UGT76G1 | MNWQILKEILGKMIKQTKASSGVIWNSFKELEESELETVIREIPAPSFLIPLPKHLTASSSSLLDHDRTVFQWLDQQP |
| CP2 | PSSVLYVSFGSTSEVDEKDFLEIARGLVDSKQSFLWVVRPGFVKGSTWVEPLPDGFLGERGRIVKWVPQQEVLAH |
| Amino | GAIGAFWTHSGWNSTLESVCEGVPMIFSDFGLDQPLNARYMSDVEKVGVYLENGWERGEIANAIRRVMVDEEGE |
| Acid | YIRQNARVLKQKADVSLMKGGSSYESLESLVSYISSLYKDDSGYSSSYAAAAGMENKTETTVRRRRRIILFPVPFQ |
| | GHINPILQLANVLYSKGFSITIFHTNFNKPKTSNYPHFTFRFILDNDPQDERISNLPTHGPLAGMRIPIINEHGADELRR |
| | ELELLMLASEEDELVSCLITDALWYFAQSVADSLNLRRLVLMTSSLFNFHAHVSLPQFDELGYLDPDDKTRLELQA |
| | SGFPMLKVKDIKSAYS (SEQ ID NO: 4) |
| EUGT11 | MDSGYSSSYAAAAGMHVVICPWLAFGHLLPCLDLAQRLASRGHRVSFVSTPRNISRLPPVRPALAPLVAFVALPLP |
| WT | RVEGLPDGAESTNDVPHDRPDMVELHRRAFDGLAAPFSEFLGTACADWVIVDVFHHWAAAAALEHKVPCAMML |
| Amino | LGSAHMIASIADRRLERALTESPAAAGQGRPAAAPTFEVARMKLIRTKGSSGMSLAERFSLTLSRSSLVVGRSCVEF |
| Acid | EPETVPLLSTLRGKPITFLGLMPPLHEGRREDGEDATVRWLDAQPAKSVVYVALGSEVPLGVEKVHELALGLELA |
| | GTRFLWALRKPTGVSDADLLPAGFEERTRGRGVVATRWVPQMSILAHAAVGAFLTHCGWNSTIEGLMFGHPLIM |
| | LPIFGDQGPNARLIEAKNAGLQVARNDGDGSFDREGVAAAIRAVAVELESSKVFQAKAKKLQEIVADMACHERYI |
| | DGFIQQLRSYKD (SEQ ID NO: 5) |
| EUGT11 | MGSSGMSLALRFSLTLSRSSLVVGRSCVEFEPETVPLLSTLRGKPITFLGLMPPLHEGRREDGEDATVRWLDAQPA |
| CP1 | KSVVYVALGSEVPLGVEKVHELALGLELAGTRFLWALRKPTGVSDADLLPAGFEERTRGRGVVATRWVPQMSIL |
| Amino | AHAAVGAFLTHCGWNSTIEGLMFGHPLIMLPIFGDQGPNARLIEAKNAGLQVARNDGDGSFDREGVAAAIRAVAV |
| Acid | ELESSKVFQAKAKKLQEIVADMACHERYIDGFIQQLRSYKDDSGYSSSYAAAAGMHVVICPWLAFGHLLPCLDLA |
| | QRLASRGHRVSFVSTPRNISRLPPVRPALAPLVAFVALPLPRVEGLPDGAESTNDVPHDRPDMVELHRRAFDGLAA |
| | PFSEFLGTACADWVIVDVFHHWAAAAALEHKVPCAMMLLGSAHMIASIADRRLERALTESPAAAGQGRPAAAPT |
| | FEVARMKLIRTK (SEQ ID NO: 6) |
| HV1 | MDGNSSSSPLHVVICPWLALGHLLPCLDIALRLASRGHRVSFVSTPRNIARLPPLRPAVAPLVDFVALPLPHVDGLP |
| glycosyl- | EGAESTNDVPYDKFELHRKAFDGLAAPFSEFLRAACAEGAGSRPDWLIVDTFHHWAAAAAVENKVPCVMLLLGA |
| transferase | ATVIAGFARGVSEHAAAAVGKERPAAEAPSFETERRKLMTTQNASGMTVAERYFLTLMRSDLVAIRSCAEWEPES |
| amino acid | VAALTTLAGKPVVPLGLLPPSPEGGRGVSKEDAAVRWLDAQPAKSVVYVALGSEVPLRAEQVHELALGLELSGA |
| | RFLWALRKPTDAPDAAVLPPGFEERTRGRGLVVTGWVPQIGVLAHGAVAAFLTHCGWNSTIEGLLFGHPLIMLPIS |
| | SDQGPNARLMEGRKVGMQVPRDESDGSFRREDVAATVRAVAVEEDGRRVFTANAKKMQEIVADGACHERCIDG |
| | FIQQLRSYKA (SEQ ID NO: 7) |
| SUSI WT | MANAERMITRVHSQRERLNETLVSERNEVLALLSRVEAKGKGILQQNQIIAEFEALPEQTRKKLEGGPFFDLLKSTQ |
| from | EAIVLPPWVALAVRPRPGVWEYLRVNLHALVVELLQPAEFLHFKEELVDGVKNGNFTLELDFEPFNASIPRPTLHK |
| Arabidopsis | YIGNGVDFLNRHLSAKLFHDKESLLPLLKFLRLHSHQGKNLMLSEKIQNLNTLQHTLRKAEEYLAELKSETLYEEF |
| thaliana, | EAKFEEIGLERGWGDNAERVLDMIRLLLDLLEAPDPCTLETFLGRVPMVFNVVILSPHGYFAQDNVLGYPDTGGQ |
| Amino | VVYILDQVRALEIEMLQRIKQQGLNIKPRILILTRLLPDAVGTTCGERLERVYDSLYCDILRVPFRTEKGIVRKWISR |
| Acid | FEVWPYLETYTEDAAVELSKELNGKPDLIIGNYSDGNLVASLLAHKLGVTQCTIAHALEKTKYPDSDIYWKKLDD |
| | KYHFSCQFTADIFAMNHTDFIITSTFQEIAGSKETVGQYESHTAFTLPGLYRVVHGIDVFDPKFNIVSPGADMSIYFP |
| | YTEEKRRLTKFHSEILELLYSDVENKEHLCVLKDKKKPILFTMARLDRVKNLSGLVEWYGKNTRLRELANLVVVG |
| | GDRRKESKDNEEKALMKKMYDLIEEYKLNGQFRWISSQMDRVRNGELYRYICDTKGAFVQPALYEAFGLTVVEA |
| | MTCGLPTFATCKGGPAEIIVHGKSGFHIDPYHGDQAADTLADFFTKCKEDPSHWDEISKGGLQRIEEKYTWQIYSQ |
| | RLLTLTGVYGFWKHVSNLDRLEARRYLEMFYALKYRPLAQAVPLAQDD (SEQ ID NO: 8) |
| UGT76G1- | MENKTETTVRRRRRIILFEPVPFQGHINPILQLANVLYSKGFSITIFHTNFNKPKTSNYPHFTFRFILDNDPQDERISNLP |
| AtSUS1 | THGPLAGMRIPIINEHGADELRRELELLMLASEEDELVSCLITDALWYFAQSVADSLNLRRLVLMTSSLFNFHAHVS |
| fusion | LPQFDELGYLDPDDKTRLEEQASGFPMLKVKDIKSAYSNWQILKEILGKMIKQTKASSGVIWNSFKELLESELETVI |
| enzyme, | REIPAPSFLIPLPKHLTASSSSLLDHDRTVFQWLDQQPPSSVLYVSFGSTSEVDEKDFLEIARGLVDSKQSFLWVVRP |
| amino acid | GFVKGSTWVEPLPDGFLGERGRIVKWVPQQEVLAHGAIGAFWTHSGWNSTLESVCEGVPMIFSDFGLDQPLNARY |
| | MSDVLKVGVYLENGWERGEIANAIRRVMVDEEGEYIRQNARVLKQKADVSLMKGGSSYESLESLVSYISSLGSGA |
| | NAERMITRVHSQRERLNETLVSERNEVLALLSRVEAKGKGILQQNQIIAEFEALPEQTRKKLEGGPFFDLLKSTQEA |
| | IVLPPWVALAVRPRPGVWEYLRVNLHALVVELLQPAEFLHFKEELVDGVKNGNFTLELDEEPFNASIPRPTLHKYI |
| | GNGVDFLNRHLSAKLFHDKESLLPLLKFLRLHSHQGKNLMLSEKIQNLNTLQHTLRKAEEYLAELKSETLYEEFEA |
| | KFEEIGLERGWGDNAERVLDMIRLLLDLLEAPDPCTLETELGRVPMVFNVVILSPHGYFAQDNVLGYPDTGGQVV |
| | YILDQVRALEIEMLQRIKQQGLNIKPRILILTRLLPDAVGTTCGERLERVYDSEYCDILRVPFRTEKGIVRKWISRFEV |
| | WPYLETYTEDAAVELSKELNGKPDLIIGNYSDGNLVASLLAHKLGVTQCTIAHALEKTKYPDSDIYWKKLDDKYH |
| | FSCQFTADIFAMNHTDFIITSTFQEIAGSKETVGQYESHTAFTLPGLYRVVHGIDVFDPKFNIVSPGADMSIYFPYTEE |
| | KRRLTKFHSEILEELYSDVENKEHLCVLKDKKKPILFTMARLDRVKNLSGLVEWYGKNTRLRELANLVVVGGDRR |
| | KESKDNEEKALMKKMYDLIEEYKLNGQFRWISSQMDRVRNGELYRYICDTKGAFVQPALYEAFGLTVVEAMTCG |
| | LPTFATCKGGPAEIIVHGKSGFHIDPYHGDQAADTLADFFTKCKEDPSHWDEISKGGLQRIEEKYTWQIYSQRLLTL |
| | TGVYGFWKHVSNLDRLEARRYLEMFYALKYRPLAQAVPLAQDDWT (SEQ ID NO: 9) |
| EUGT11- | MDSGYSSSYAAAAGMHVVICPWLAFGHLLPCLDLAQRLASRGHRVSFVSTPRNISRLPPVRPALAPLVAFVALPLP |
| AtSUS1 | RVEGLPDGAESTNDVPHDRPDMVELHRRAFDGLAAPFSEFLGTACADWVIVDVFHHWAAAAALEHKVPCAMML |
| fusion | LGSAHMIASIADRRLERAETESPAAAGQGRPAAAPTFEVARMKLIRTKGSSGMSLAERFSLTLSRSSLVVGRSCVEF |
| enzyme, | EPETVPLLSTLRGKPITFLGLMPPLHEGRREDGEDATVRWLDAQPAKSVVYVALGSEVPLGVEKVHELALGLELA |
| amino acid | GTRFLWALRKPTGVSDADLLPAGFEERTRGRGVVATRWVPQMSILAHAAVGAFLTHCGWNSTIEGLMFGHPLIM |
| | LPIFGDQGPNARLIEAKNAGLQVARNDGDGSFDREGVAAAIRAVAVELESSKVFQAKAKKLQEIVADMACHERYI |
| | DGFIQQLRSYKDGSGANAERMITRVHSQRERLNETLVSERNEVLALLSRVEAKGKGILQQNQIIAEFEALPEQTRKK |
| | LEGGPFFDLLKSTQEAIVLPPWVALAVRPRPGVWEYLRVNLHALVVEELQPAEFLHFKEELVDGVKNGNFTLELD |
| | FEPFNASIPRPTLHKYIGNGVDFLNRHLSAKLFHDKESLLPLLKFLRLHSHQGKNLMLSEKIQNLNTLQHTLRKAEE |
| | YLAELKSETLYEEFEAKFEEIGLERGWGDNAERVLDMIRLLLDLLEAPDPCTLETFLGRVPMVFNVVILSPHGYFA |
| | QDNVLGYPDTGGQVVYILDQVRALEIEMLQRIKQQGLNIKPRILILTRLLPDAVGTTCGERLERVYDSEYCDILRVP |
| | FRTEKGIVRKWISRFEVWPYLETYTEDAAVELSKELNGKPDLIIGNYSDGNLVASLLAHKLGVTQCTIAHALEKTK |
| | YPDSDIYWKKLDDKYHFSCQFTADIFAMNHTDFIITSTFQEIAGSKETVGQYESHTAFTLPGLYRVVHGIDVFDPKE |
| | NIVSPGADMSIYFPYTEEKRRLTKFHSEILELLYSDVENKEHLCVLKDKKKPILFTMARLDRVKNLSGLVEWYGKN |
| | TRLRELANLVVVGGDRRKESKDNEEKAEMKKMYDLIEEYKLNGQFRWISSQMDRVRNGELYRYICDTKGAFVQP |
| | ALYEAFGLTVVEAMTCGLPTFATCKGGPAEIIVHGKSGFHIDPYHGDQAADTLADFFTKCKEDPSHWDEISKGGLQ |
| | RIEEKYTWQIYSQRLLTLTGVYGFWKHVSNLDRLEARRYLEMFYALKYRPLAQAVPLAQDD (SEQ ID NO: 10) |
| HV1- | MDGNSSSSPLHVVICPWLALGHLLPCLDIAERLASRGHRVSFVSTPRNIARLPPLRPAVAPLVDFVALPLPHVDGLP |
| AtSUS1 | EGAESTNDVPYDKFELHRKAFDGLAAPFSEFLRAACAEGAGSRPDWLIVITFHHWAAAAAVENKVPCVMLLLGA |
| fusion | ATVIAGFARGVSEHAAAAVGKERPAAEAPSFETERRKLMTTQNASGMTVAERYFLTLMRSDLVAIRSCAEWEPES |
| enzyme, | VAALTTLAGKPVVPLGLLPPSPEGGRGVSKEDAAVRWLDAQPAKSVVYVALGSEVPLRAEQVHELALGLELSGA |
| amino acid | RFLWALRKPTDAPDAAVLPPGFEERTRGRGLVVTGWVPQIGVLAHGAVAAFLTHCGWNSTIEGLLFGHPLIMLPIS |
| | SDQGPNARLMEGRKVGMQVPRDESDGSFRREDVAATVRAVAVEEDGRRVFTANAKKMQEIVADGACHERCIDG |
| | FIQQLRSYKAGSGANAERMITRVHSQRERLNETLVSERNEVLALLSRVEAKGKGILQQNQIIAEFEALPEQTRKKLE |
| | GGPFFDLLKSTQEAIVLPPWVALAVRPRPGVWEYLRVNLHALVVEELQPAEFLHFKEELVDGVKNGNFTLELDFE |
| | PFNASIPRPTLHKYIGNGVDFLNRHLSAKLFHDKESLLPLLKFLRLHSHQGKNLMLSEKIQNLNTLQHTLRKAEEYL |
| | AELKSETLYEEFEAKFEEIGLERGWGDNAERVLDMIRLLLDLLEAPDPCTLETFLGRVPMVFNVVILSPHGYFAQD |
| | NVLGYPDTGGQVVYILDQVRALEIEMLQRIKQQGLNIKPRILILTRLLPDAVGTTCGERLERVYDSEYCDILRVPFR |
| | TEKGIVRKWISRFEVWPYLETYTEDAAVELSKELNGKPDLIIGNYSDGNLVASLLAHKLGVTQCTIAHALEKTKYP |
| | DSDIYWKKLDDKYHFSCQFTADIFAMNHTDFIITSTFQEIAGSKETVGQYESHTAFTLPGLYRVVHGIDVFDPKFNI |
| | VSPGADMSIYFPYTEEKRRLTKFHSEIEELLYSDVENKEHLCVLKDKKKPILFTMARLDRVKNLSGLVEWYGKNTR |
| | LRELANLVVVGGDRRKESKDNEEKAEMKKMYDLIEEYKLNGQFRWISSQMDRVRNGELYRYICDTKGAFVQPAL |
| | YEAFGLTVVEAMTCGLPTFATCKGGPAEIIVHGKSGFHIDPYHGDQAADTLADFFTKCKEDPSHWDEISKGGLQRI |
| | EEKYTWQIYSQRLLTLTGVYGFWKHVSNLDRLEARRYLEMFYALKYRPLAQAVPLAQDD (SEQ ID NO: 11) |

### Ferulic Acid

Ferulic acid is a hydrocinnamic acid that is abundant in plant cells, particularly as a component of pectin, lignin, and plant cell walls. In nature, ferulic acid serves as a precursor for the synthesis of many other organic compounds. It is frequently found in edible plants such as corn, rice, bamboo shoots, cereals, flaxseed, tea leaves, and legumes. Ferulic acid is common in plant-based foods such as rice bran oil, breads containing flaxseed, and rye bread. Ferulic acid acts as an antioxidant, and can therefore protect the Rebaudiosides with which it is formulated from oxidation, which results in undesired off-flavors. Methods of producing ferulic acid are described in U.S. Pat. No. 5,288,902, which methods are incorporated herein by reference. A brief description, which provides an overview of exemplary methods is provided.

Briefly, U.S. Pat. No. 5,288,902 provides a method of manufacturing ferulic acid by hydrolyzing oryzanol, which method is incorporated by reference. Briefly, waste material, by-product or a crude oryzanol can be subjected to hydrolysis in the presence of an alkali so as to manufacture ferulic acid. In general, sodium hydroxide or potassium hydroxide can be used as an alkali in the step of hydrolyzing oryzanol. It is also possible to use other alkaline compounds such as LiOH, RuOH, Na₂ CO₃, K₂ CO₃ and NaHCO₃. In the next step, a solution containing an alkali salt of ferulic acid can be acidified with, for example, a dilute sulfuric acid so as to precipitate ferulic acid in the solution, followed by separating the precipitated ferulic acid by mean of filtration. The crude ferulic acid separated by filtration can be dissolved in hot water (about 90 to 100° C.) and, then, the system can be cooled so as to permit precipitation of ferulic acid. A pure trans-ferulic acid can be obtained, which refers to the naturally occurring form of ferulic acid.

### Orally Consumable Products

Any one of the formulations described herein can be used for the production of baked goods, dairy products, spreads, margarines, sports products, nutrition bars and infant formulas, feed, aquaculture, nutraceuticals and medicinal products.

In some embodiments, any one of the formulations described herein may be used for creating or enhancing a sweetening effect of an orally consumable products. In some embodiments, methods of creating or enhancing a sweetening effect of an orally consumable product comprises adding an amount of any one of the formulations described herein sufficient to produce the desired degree of sweetness to the orally consumable product.

Accordingly, other aspects of the present disclosure provide orally consumable products comprising any one of the formulations described herein. In some embodiments, the orally consumable product is selected from the group consisting of a food composition, a beverage product, a dietary supplement, a nutraceutical, an edible gel mix, an edible gel composition, a pharmaceutical composition, a dental and oral hygiene composition, and an animal feed.

In some embodiments, the orally consumable product comprising any one of the formulations described herein is a dental and oral hygiene composition. Examples of suitable dental and oral hygiene compositions can be, for example, toothpastes, tooth polishes, dental floss, mouthwashes, mouth rinses, dentrifices, mouth sprays, mouth refreshers, plaque rinses, dental pain relievers, and the like. In some embodiments, the dental and oral hygiene composition is a toothpaste.

In some embodiments, the orally consumable product comprising any one of the formulations described herein is a pharmaceutical composition. In some embodiments, the pharmaceutical composition comprises any one of the formulations described herein, and further comprises one or more pharmaceutically acceptable excipients. In some embodiments, pharmaceutical compositions of the present disclosure can be used to formulate pharmaceutical drugs containing one or more active agents that exert a biological effect. Accordingly, in some embodiments, pharmaceutical compositions of the present disclosure can contain one or more active agents that exert a biological effect. Suitable active agents are well known in the art (e.g., The Physician's Desk Reference). Such compositions can be prepared according to procedures well known in the art, for example, as described in Remington's Pharmaceutical Sciences, Mack Publishing Co., Easton, Pa., USA. In an embodiment of any one of the methods or compositions provided herein, the pharmaceutical composition comprises any one of the formulations described herein and a pharmaceutical, and is in an encapsulated form. In an embodiment of any one of the methods or compositions provided herein, the pharmaceutical composition comprises any one of the formulations described herein and a pharmaceutical, and is in a liquid or gel that is encapsulated. Encapsulation can be achieved by spray drying, oven dying, glass encapsulation, etc.

In some embodiments, the orally consumable product comprising any one of the formulations described herein is a beverage (e.g., a carbonated beverage product or a non-carbonated beverage product). The beverage can also be, for example, a soft drink, a fountain beverage, a frozen beverage; a ready-to-drink beverage; a frozen and ready-to-drink beverage, coffee, tea, a dairy beverage, a powdered soft drink, a liquid concentrate, flavored water, enhanced water, fruit juice, a fruit juice flavored drink, a sport drink, or an energy drink, isotonic drinks, low-calorie drinks, zero-calorie drinks, vegetable juices, juice drinks, dairy drinks, yoghurt drinks, alcohol beverages, protein beverages, dairy beverages, or plant-based beverages. In an embodiment, any one of the beverages as described herein can be in powdered form (i.e., a powdered beverage) and may be reconstituted subsequently, such as by a consumer. In an embodiment, any one of the beverages as described herein can be in liquid form.

In some embodiments, the beverage of the present disclosure comprises any one of the formulations described herein, and further comprises one or more beverage ingredients such as, for example, acidulants, fruit juices and/or vegetable juices, pulp, etc., flavorings, coloring, preservatives, vitamins, minerals, electrolytes, erythritol, tagatose, glycerine, and carbon dioxide. The beverages described herein may be provided in any suitable form, such as a beverage concentrate and a carbonated, ready-to-drink beverage.

In certain embodiments, the beverages of the present disclosure can have any of numerous different specific formulations or constitutions. The formulation of a beverage of the present disclosure can vary to a certain extent, depending upon such factors as the product's intended market segment, its desired nutritional characteristics, flavor profile, and the like. For example, in certain embodiments, it can generally be an option to add further ingredients to the formulation of a particular beverage product. For example, additional (i.e., more and/or other) sweeteners can be added, flavorings, electrolytes, vitamins, fruit juices or other fruit products, tastents, masking agents and the like, flavor enhancers, and/or carbonation typically may be added to any such formulations to vary the taste, mouthfeel, nutritional characteristics, etc.

In some embodiments, the orally consumable product comprising any one of the formulations described herein is a food composition. A “food composition” refers to any solid or liquid ingestible material that can, but need not, have a nutritional value and be intended for consumption by humans and animals. Examples of suitable food product compositions can be, for example, confectionary compositions, such as candies, mints, fruit flavored drops, cocoa products, chocolates, and the like; condiments, such as ketchup, mustard, mayonnaise, and the like; chewing gums; cereal compositions; baked goods, such as breads, cakes, pies, cookies, and the like; dairy products, such as milk, cheese, cream, ice cream, sour cream, yogurt, sherbet, and the like; tabletop sweetener compositions; soups; stews; convenience foods; meats, such as ham, bacon, sausages, jerky, and the like; gelatins and gelatin-like products such as jams, jellies, preserves, and the like; fruits; vegetables; egg products; icings; syrups including molasses; snacks; nut meats and nut products; and animal feed. Other non-limiting examples of food compositions include bakery products, cookies, biscuits, baking mixes, cereals, confectioneries, candies, toffees, chewing gum, dairy products, flavored milk, yoghurts, flavored yoghurts, cultured milk, soy sauce and other soy base products, salad dressings, mayonnaise, vinegar, frozen-desserts, meat products, fish-meat products, bottled and canned foods, tabletop sweeteners, fruits and vegetables, herbs, spices and seasonings, natural and synthetic flavors, and flavor enhancers, such as monosodium glutamate, prepared packaged products, such as dietetic sweeteners, liquid sweeteners, granulated flavor mixes, pet foods, livestock feed, tobacco, and materials for baking applications, such as powdered baking mixes for the preparation of breads, cookies, cakes, pancakes, donuts and the like.

In some embodiments, the food composition is selected from the group consisting of spreads, margarines, sports products, nutrition bars, infant formulas, mayonnaise, confectionary composition, a condiment, a chewing gum, a cereal composition, a baked good, a dairy product, and a tabletop sweetener composition. In some embodiments, the food composition is a food composition included in Table 1. In some embodiments, the food composition is a yogurt. In some embodiments, the food composition is frozen. In some embodiments, the food composition is ice cream.

TABLE 1
Examples of orally consumable compositions

| | DAIRY | | PREPARED | OIL BASED | |
|---|---|---|---|---|---|
| BEVERAGES | PRODUCTS | BAKING | FOODS | PRODUCTS | SNACK FOODS |
| Soy milks | Cheeses | Breads | Entrees | Salad | Granola |
| Smoothies | Cream | Rolls | Side Dishes | Dressing | Cereals |
| Fruit Juices | Cheeses | Cakes | Soups | Mayonnaise | Snack/Nutritional |
| Dairy | Sour Cream | Pastries | Sauces | Margarine/ | Bars |
| Drinks | Yogurt | Cookies | Processed | Spreads | Confectionary |
| | Yogurt | Crackers | Meats | Shortening | |
| | Drinks | Muffins | Processed | | |
| | Non-Dairy | | Fish | | |
| | Creamers | | Pet Foods | | |
| | Dips | | | | |

Food compositions described herein include any preparations or compositions which are suitable for consumption and are used for nutrition or enjoyment purposes. They are generally products which are intended to be eaten by humans or animals and introduced into the body through the mouth, to remain there for a certain time and then either be eaten (e.g. ready-to-eat foodstuffs or feeds, see also herein below) or removed (e.g. chewing gums). Such products include any substances or products which in the processed, partially processed or unprocessed state are to be ingested by humans or animals. They also include substances which are added to orally consumable products during their manufacture, preparation or treatment and which are intended to be introduced into the human or animal oral cavity.

The food compositions according to the disclosure also include substances which in the unchanged, treated or prepared state are to be swallowed by a human or animal and then digested; in this respect, the orally consumable products according to the disclosure also include casings, coatings or other encapsulations which are to be swallowed at the same time or which may be expected to be swallowed. The expression “food composition” covers ready-to-eat foodstuffs, beverages and feeds, that is to say foodstuffs, beverages or feeds that are already complete in terms of the substances that are important for the taste. The expressions “ready-to-eat foodstuff” and “ready-to-eat feed” also include drinks as well as solid or semi-solid ready-to-eat foodstuffs or feeds. Examples which may be mentioned are frozen products, which must be thawed and heated to eating temperature before they are eaten. Products such as yoghurt or ice-cream as well as chewing gums or hard caramels are also included among the ready-to-eat foodstuffs or feeds of the current disclosure.

In some embodiments, the orally consumable product comprising any one of the formulations described herein is an animal feed product for livestock, companion animals and/or aquaculture. In some embodiments, the livestock is cattle, swine and/or poultry. In some embodiments, the animal feed product further comprises a hydrocolloid or erythritol.

In some embodiments, any one of the orally consumable products described herein further comprises a component selected from the group consisting of sucrose, aroma compounds, flavoring compounds and mixtures thereof. In some embodiments, any one of the orally consumable products described herein further comprises tocopherols in an amount of at least about 5 ppm. In some embodiments, any one of the orally consumable products described herein further comprises at least one stabilizing agent selected from the group consisting of citric acid, sodium benzoate, t-butyl hydroquinone, ascorbyl palmitate, propyl gallate, and combinations thereof. In some embodiments, any one of the orally consumable products described herein further comprises a moisture containing ingredient. In some embodiments, the moisture ingredient is an emulsion. In some embodiments, any one of the orally consumable products described herein further comprises a chelating agent.

In some embodiments, any one of the orally consumable products described herein can also have at least one additional sweetener. The at least one additional sweetener can be a natural high intensity sweetener, for example. The additional sweetener can be selected from a Stevia extract, a steviol glycoside, stevioside, rebaudioside B, rebaudioside C, rebaudioside D, rebaudioside F, dulcoside A, rubusoside, steviolbioside, sucrose, high fructose corn syrup, fructose, glucose, xylose, arabinose, rhamnose, erythritol, xylitol, mannitol, sorbitol, inositol, AceK, aspartame, neotame, sucralose, saccharine, naringin dihydrochalcone (NarDHC), neohesperidin dihydrochalcone (NDHC), rubusoside, mogroside IV, siamenoside I, mogroside V, monatin, thaumatin, monellin, brazzein, L-alanine, glycine, Lo Han Guo, hernandulcin, phyllodulcin, trilobtain, and combinations thereof. In some embodiments, any one of the orally consumable products described herein does not have a sweetener in addition to a formulation provided herein.

In some embodiments, any one of the orally consumable products described herein can also have at least one additive. The additive can be, for example, a carbohydrate, a polyol, an amino acid or salt thereof, a polyamino acid or salt thereof, a sugar acid or salt thereof, a nucleotide, an organic acid, an inorganic acid, an organic salt, an organic acid salt, an organic base salt, an inorganic salt, a bitter compound, a flavorant, a flavoring ingredient, an astringent compound, a protein, a protein hydrolysate, a surfactant, an emulsifier, a flavonoids, an alcohol, a polymer, and combinations thereof.

As used herein, “dietary supplement(s)” refers to compounds intended to supplement the diet and provide nutrients, such as vitamins, minerals, fiber, fatty acids, amino acids, etc. that may be missing or may not be consumed in sufficient quantities in a diet. Any suitable dietary supplement known in the art may be used. Examples of suitable dietary supplements can be, for example, nutrients, vitamins, minerals, fiber, fatty acids, herbs, botanicals, amino acids, and metabolites.

As used herein, “nutraceutical(s)” refers to compounds, which includes any food or part of a food that may provide medicinal or health benefits, including the prevention and/or treatment of disease or disorder (e.g., fatigue, insomnia, effects of aging, memory loss, mood disorders, cardiovascular disease and high levels of cholesterol in the blood, diabetes, osteoporosis, inflammation, autoimmune disorders, etc.). Any suitable nutraceutical known in the art may be used. In some embodiments, nutraceuticals can be used as supplements to food and beverages and as pharmaceutical formulations for enteral or parenteral applications which may be solid formulations, such as capsules or tablets, or liquid formulations, such as solutions or suspensions.

In some embodiments, dietary supplements and nutraceuticals can further contain protective hydrocolloids (such as gums, proteins, modified starches), binders, film-forming agents, encapsulating agents/materials, wall/shell materials, matrix compounds, coatings, emulsifiers, surface active agents, solubilizing agents (oils, fats, waxes, lecithins, etc.), adsorbents, carriers, fillers, co-compounds, dispersing agents, wetting agents, processing aids (solvents), flowing agents, taste-masking agents, weighting agents, jellyfying agents, gel-forming agents, antioxidants and antimicrobials.

As used herein, a “gel” refers to a colloidal system in which a network of particles spans the volume of a liquid medium. Although gels mainly are composed of liquids, and thus exhibit densities similar to liquids, gels have the structural coherence of solids due to the network of particles that spans the liquid medium. For this reason, gels generally appear to be solid, jelly-like materials. Gels can be used in a number of applications. For example, gels can be used in foods, paints, and adhesives. Gels that can be eaten are referred to as “edible gel compositions.” Edible gel compositions typically are eaten as snacks, as desserts, as a part of staple foods, or along with staple foods. Examples of suitable edible gel compositions can be, for example, gel desserts, puddings, jams, jellies, pastes, trifles, aspics, marshmallows, gummy candies, and the like. In some embodiments, edible gel mixes generally are powdered or granular solids to which a fluid may be added to form an edible gel composition. Examples of suitable fluids can be, for example, water, dairy fluids, dairy analogue fluids, juices, alcohol, alcoholic beverages, and combinations thereof. Examples of suitable dairy fluids can be, for example, milk, cultured milk, cream, fluid whey, and mixtures thereof. Examples of suitable dairy analogue fluids can be, for example, soy milk and non-dairy coffee whitener.

As used herein, the term “gelling ingredient” refers to any material that can form a colloidal system within a liquid medium. Examples of suitable gelling ingredients can be, for example, gelatin, alginate, carageenan, gum, pectin, konjac, agar, food acid, rennet, starch, starch derivatives, and combinations thereof. It is well known to those in the art that the amount of gelling ingredient used in an edible gel mix or an edible gel composition can vary considerably depending on a number of factors such as, for example, the particular gelling ingredient used, the particular fluid base used, and the desired properties of the gel.

Gel mixes and gel compositions of the present disclosure can be prepared by any suitable method known in the art. In some embodiments, edible gel mixes and edible gel compositions of the present disclosure can be prepared using other ingredients in addition to the gelling agent. Examples of other suitable ingredients can be, for example, a food acid, a salt of a food acid, a buffering system, a bulking agent, a sequestrant, a cross-linking agent, one or more flavors, one or more colors, and combinations thereof.

In certain embodiments that can be combined with any of the preceding embodiments, the orally consumable products can further include one or more additives selected from a carbohydrate, a polyol, an amino acid or salt thereof, a poly-amino acid or salt thereof, a sugar acid or salt thereof, a nucleotide, an organic acid, an inorganic acid, an organic salt, an organic acid salt, an organic base salt, an inorganic salt, a bitter compound, a flavorant, a flavoring ingredient, an astringent compound, a protein, a protein hydrolysate, a surfactant, an emulsifier, a flavonoids, an alcohol, a polymer, and combinations thereof.

The compositions can be used “as-is” or in combination with other sweeteners, flavors and food ingredients. For use in domestic applications, particularly as a replacement for sugar in beverage sweetening, it is desirable in some embodiments that the compositions according to the present disclosure include a bulking agent so that an equivalent sweetness to that provided by, for example, a teaspoonful of sugar is provided by an amount which can conveniently be handled. Any suitable soluble and edible material can be used, for example, a carbohydrate such as sucrose itself, especially transformed sugar of low density, dextrose, or sorbitol or a dextrin such as spray-dried maltodextrin. While the substances will add to the caloric value of the composition, the total will still be considerably smaller than that of the amount of sugar providing an equivalent sweetness. Alternatively, the sweetening composition may be prepared in a tablet form.

Compositions provided herein are usually stable at pH values in the range of from 2 to 10, especially 3 to 8. Dry compositions, such as powders, granules or tablets can be stable indefinitely when stored under dry conditions at room temperature. Compositions in the form of aqueous solutions can be stable indefinitely when frozen. If a preservative such as benzoic acid or its salts, sulphur dioxide or sodium meta-bisulphite is added to such a composition, it may be stored almost indefinitely at room temperature. The compositions therefore can have a long shelf-life when incorporated into soft drinks or fruit juices, or other similar food compositions containing preservatives. The limitation on the use of sugar may also positively contribute to the long shelf-life of the products provided herein.

Food compositions comprising the inventive formulations provided herein may further comprise components selected from the group consisting of additional sweeteners or sweet-tasting compounds, aroma compounds, flavoring compounds, and their mixtures. Such additives may also specifically include hydrocolloids such as pectins, gelatin, carrageenan, or gums (Arabic, guar, locust bean) for dressings, jams, jellies, confections and the like. Other additives to food, feed or beverage compositions include chelating agents whose addition is designed to protect against enzymatic reactions and may specifically include ethylenediaminetetraacetic acid (EDTA).

Aroma compounds and flavor enhancing agents are well known in the art can be added to the compositions provided herein. These flavoring agents can be chosen from synthetic flavoring liquids and/or oils derived from plants leaves, flowers, or fruits. Representative flavoring liquids include: artificial, natural or synthetic fruit flavors such as eucalyptus, lemon, orange, banana, grape, lime, apricot, and grapefruit oils, fruit essences including apple, strawberry, cherry, orange, pineapple, and so forth, bean- and nut-derived flavors such as coffee, cocoa, cola, hazelnut, peanut or almond, and root-derived flavors such as licorice or ginger.

The following examples illustrate various embodiments of the invention. It will be understood that the invention is not limited to the materials, proportions, conditions and procedures set forth in the examples, which are only illustrative.

### EXAMPLES

Exemplary formulations containing Reb E and Reb I and ferulic acid are shown in Table 2.

TABLE 2

| | Usage/100 mL | Usage/1 kg | Usage/100 kg |
|---|---|---|---|
| | Batch (g) | Batch (g) | Batch (kg) |
| Reb E 95% | 0.0045 | 15 | 1.50 |
| Reb I 95% | 0.003 | 10 | 1.00 |
| Ferulic acid | 0.0012 | 4 | 0.4 |
| 98% | | | |
| Propylene glycol | 0.2913 | 971 | 97.10 |

The formulation containing Reb E and Reb I and ferulic acid may be formulated in a liquid. Propylene glycol and/or SM00056 may be used as a carrier with any one of the formulations, such as a carrier in a liquid composition. Maltodextrin may be used as a carrier with any one of the formulations, such as a carrier in a powder composition.

A liquid formulation can be prepared by the following process:
1) Heat propylene glycol to 120° F.
2) Add Reb E and Reb I until dispersed.
3) Add ferulic acid until dispersed.
4) Mix liquid until flavor is clear.
5) Cool to <75° F.

A powder can be prepared by the following process:
1) Dissolve ferulic acid in alcohol.
2) Combine ferulic acid solution with medium-chain triglyceride (MCT) oil.
3) Separately, mix maltodextrin and gum arabic in water.
4) Combine ferulic acid-alcohol-oil mixture with maltodextrin-gum arabic mixture, homogenize at pressure of 2,000 pounds per square inch (psi).
5) Spray homogenized ferulic acid mixture with inlet temperature of 380° F. and outlet temperature of 180° F.
6) Mix spray-dried ferulic acid with powdered Reb E and Reb I until uniform.

1. A composition comprising Reb E, Reb I, and ferulic acid.

2. A composition comprising Reb E, Reb I, and ferulic acid, wherein Reb I is present in the composition in an amount of from about 2 ppm to about 30 ppm.

3. The composition of claim 1, wherein Reb E is present in the composition in an amount of from about 3 ppm to about 45 ppm.

4. A composition comprising Reb E, Reb I, and ferulic acid, wherein ferulic acid is present in the composition in an amount of from about 0.8 ppm to about 30 ppm.

5. The composition of claim 4, wherein ferulic acid is present in the composition in an amount of from about 0.8 ppm to about 12 ppm.

6.-7. (canceled)

8. The composition of claim 5, wherein Reb E is present in the composition in an amount of from about 3 ppm to about 45 ppm, and wherein Reb I is present in the composition in an amount of from about 2 ppm to about 30 ppm.

9. The composition of claim 1, wherein the composition comprises 3-45 ppm Reb E, 2-30 ppm Reb I, and 0.8-30 ppm ferulic acid.

10. (canceled)

11. The composition of claim 1, wherein the composition comprises about 40-60 wt. % Reb E, about 25-45 wt. % Reb I, and about 5-25 wt. % ferulic acid, all percentages being on an anhydrous wt./wt. basis.

12. A consumable comprising the composition of claim 1.

13. The consumable of claim 12, wherein the consumable comprises about 0.2-0.30 wt. % the composition, on an anhydrous wt./wt. basis.

14. An oral composition comprising the composition of claim 1.

15. A pharmaceutical composition comprising the composition of claim 1 and a pharmaceutically acceptable excipient.

16. A beverage comprising the composition of claim 1.

17. A food composition comprising the composition of claim 1.

18. An animal feed product comprising the composition of claim 1.

19. A liquid comprising the composition of claim 1.

20. The liquid of claim 19, wherein the liquid further comprises a solvent.

21. (canceled)

22. The liquid of claim 20, wherein the solvent is ethyl alcohol, propylene glycol, glycerin, SM00056, or water and/or combinations thereof.

23. (canceled)

24. A powder comprising the composition of claim 1.

25. The powder of claim 24, further comprising dextrin or maltodextrin.

26.-32. (canceled)

33. A method for creating or enhancing the sweetness of a consumable, oral composition, pharmaceutical composition, beverage, food composition, animal feed product, liquid or powder, comprising adding an amount of the composition of claim 1 to produce the desired degree of sweetness for the consumable, oral composition, pharmaceutical composition, beverage, food composition, animal feed product, liquid or powder.
